(19) United States
(12) Patent Application Publication
(10) Pub. No.: US 2016/0374908 A1
(43) Pub. Date: Dec. 29, 2016

(54) SKIN CARE COMPOSITION AND METHODS OF USING THE SAME

(71) Applicant: The Procter & Gamble Company, Cincinnati, OH (US)

(72) Inventors: Tomohiro HAKOZAKI, Cincinnati, OH (US); John Erich OBLONG, Loveland, OH (US); Jesus VELAZQUEZ, Cincinnati, OH (US); Jiten Odhavji DIHORA, Liberty Township, OH (US)

(21) Appl. No.: 15/196,967

(22) Filed: Jun. 29, 2016

Related U.S. Application Data
(60) Provisional application No. 62/186,024, filed on Jun. 29, 2015.

(51) Int. Cl. A61K 8/11; A61K 8/60; A61Q 19/02; A61K 8/02

(52) U.S. Cl. CPC A61K 8/11; A61K 8/0241; A61K 8/602; A61Q 19/02; A61K 2800/412

(57) ABSTRACT

A stable cosmetic agent in the form of a plurality of encapsulated particles in a core-shell configuration. The encapsulated particles are formed by coating nicotinamide riboside powder or a nicotinamide riboside containing material with one or more water-insoluble encapsulating agents. The stable encapsulated nicotinamide riboside particles exhibit less than 20% hydrolysis when incorporated into an aqueous skin care composition.

### FIELD

The present disclosure is directed generally to a skin care composition comprising a stable skin care agent. More specifically, the present disclosure is directed to a skin care composition comprising an effective amount of encapsulated nicotinamide riboside particles.

### BACKGROUND

Skin conditions include some of the most common disorders treated in the developing world, and treating such conditions has led to a booming skin care industry that generates billions of dollars in sales each year. Different skin conditions are associated with widely varied triggers, biological mechanisms, environmental factors, and clinical manifestations. For example, as people age, intrinsic factors related to the biochemical changes within the skin typically result in visible signs of skin aging such as wrinkling and other forms of roughness (including increased pore size, flaking and skin lines) and/or uneven skin pigmentation (e.g., age spots or melasma). In some instances, lifestyle choices and exposure to the environment may allow extrinsic factors such as ultraviolet radiation, pollution (e.g., engine exhaust, cigarette smoke, smog), wind, heat, low humidity, harsh surfactants, abrasives, and the like to damage the skin, leading to undesirable skin appearance. As a result, a multitude of cosmetic skin care products have been developed that contain skin care agents tailored to treat common skin conditions.

An example of skin care agents known for use in skin care products are Vitamin B₃ compounds such as niacin and its derivatives. U.S. Pat. No. 4,096,240 refers to niacin as effective in skin lightening. U.S. Pat. No. 8,106,184 discloses treating skin or epithelial cells with a nicotinoyl riboside or derivative compound that increases the level of intracellular nicotinamide adenine dinucleotide NAD+ to treat skin afflictions or skin conditions such as disorders or diseases associated with or caused by inflammation, sun damage or natural aging. U.S. Publication No. 2005/0267023 discloses methods and compositions for modulating the life span of a cell or its resistance to stress, for example, by contacting the cell with nicotinamide riboside to stimulate the NAD+ salvage pathway in the cell. PCT Pub. No. WO 2015/066382 (“Deren-Lewis”) relates to methods of using nicotinamide riboside to promote the increase of intracellular levels of (NAD+) in cells and tissues for improving cell and tissue survival. Deren-Lewis discloses the use of topical nicotinamide riboside compositions for treating a variety of skin conditions by modulating the NAD+ pathway.

It has recently been found that nicotinamide riboside (“NR”) may be a particularly suitable skin care agent when applied topically or ingested. But incorporating NR into an aqueous cosmetic composition can be problematic. Many cosmetic compositions include water, and NR tends to hydrolyze in the presence of water. The rate and amount of hydrolysis depends on the amount of water present, the length of time the NR is exposed to the water and the temperature. See, “Kinetic a-Deuterium Isotope Effects for Enzymatic and Nonenzymatic Hydrolysis of Nicotinamide-β-Riboside” by Ferraz, et al., Department of Chemistry, Indiana University, Archives of Biochemistry and Biophysics, Vol. 191, No. 2, pp. 431-436, 1978. Thus, by the time a consumer is ready to use an NR-containing cosmetic product, the NR may be substantially degraded or no longer present. In some instances, it may even be desirable to incorporate NR into ingestible compositions such as beverages, which typically include a substantial amount of water. In these instances, it is particularly important to minimize or prevent hydrolysis of NR in the composition.

US 2012/0015004 (“Mironov”) relates to encapsulated nutrient salts for use in high-acid beverages. However, Mironov does not recognize the skin care benefits that NR can provide, nor that NR hydrolyses when incorporated into an aqueous compositions.

U.S. Pub. Nos. 2003/0207776, 2003/0232091, 2011/10268802, 2011/0269657, and 2015/0099680 disclose examples of encapsulating materials suitable for a wide variety of different uses, but none of these publications recognize the hydrolysis problem encountered when incorporating NR into an aqueous composition or the benefit of encapsulating NR to improve the stability of NR in an aqueous composition.

Accordingly, it would be desirable to provide an aqueous cosmetic composition that includes an effective and stable amount of NR.

### SUMMARY

Aqueous skin care compositions comprising stable nicotinamide riboside particles and methods of using the same are provided. In one aspect the skin care composition comprises an effective amount of encapsulated NR particles, each particle comprising at least one core surrounded by at least one shell. The core contains the nicotinamide riboside (NR) and the shell is formed from a water insoluble encapsulation agent. The particles are dispersed or suspended in a dermatologically acceptable carrier to provide the skin care composition.

In another aspect, a method of lightening skin is provided. The method comprises identifying a target portion of skin where skin lightening is desired and topically applying a skin care composition comprising an effective amount of NR to the target portion of skin during a treatment period. The length of the treatment period is sufficient to allow the NR to lighten the skin. Skin lightening may be demonstrate by a positive change in L* value, for example, of at least 0.1.

In another aspect, a method of improving the appearance of a hyperpigmented spot is provided. The method comprises identifying a target portion of skin that includes a hyperpigmented spot; and topically applying a skin care composition comprising an effective amount of NR to the target portion of skin during a treatment period. The length of the treatment period is sufficient to allow the NR to improve the appearance of the hyperpigmented spot, for example, by reducing the hyperpigmented spot area.

### BRIEF DESCRIPTION OF THE DRAWINGS

FIG. 1 is an illustration an encapsulated particle in a single core single shell configuration.

FIG. 2 is an illustration of an encapsulated particle in a single core, multiple shell configuration.

FIG. 3 is an illustration of an encapsulated particle in a multiple core, single shell configuration.

FIG. 4 is a chart showing the amount of HMGB1 released from keratinocytes exposed to UVB.

FIG. 5 shows an image of a face with a portion of the cheek masked.

### DETAILED DESCRIPTION

The susceptibility of NR to hydrolysis limits its usefulness in skin care compositions, many of which tend to be aqueous. In order to reduce and/or prevent the hydrolysis of NR in an aqueous composition, it has now been found that coating NR with an encapsulation agent improves the stability of the NR in aqueous compositions.

Materials, features, structures and/or characteristics of the encapsulated skin care agent described herein may be combined in any suitable manner across different embodiments, and materials, features, structures and/or characteristics may be omitted or substituted from what is described. Thus, embodiments and instances described herein may comprise or be combinable with elements or components of other embodiments and/or instances despite not being expressly exemplified in combination, unless otherwise stated or an incompatibility is stated.

All percentages are by weight of the cosmetic composition or encapsulated particles, as indicated, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

The cosmetic compositions herein can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About,” as used herein, modifies a particular value, by referring to a range equal to the particular value, plus or minus twenty percent (+/−20%).

“Apply” or “application”, as used in reference to a composition, means to apply or spread the compositions of the present invention onto a human skin surface such as the epidermis.

“Aqueous composition” refers to a composition that contains at least 20% water.

“Cosmetic” means providing a desired visual effect on an area of the human body. The visual cosmetic effect may be temporary, semi-permanent, or permanent.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (“GRAS”) by the U.S. Food and Drug Administration, food additives, and materials used in non-cosmetic consumer products including over-the-counter medications. The compositions herein may optionally include one or more cosmetic agents in addition to nicotinamide riboside. In some embodiments, cosmetic agents may be incorporated in a cosmetic composition comprising a dermatologically acceptable carrier suitable for topical application to skin.

“Dendricity” means the total length of dendrites measured on one or more melanocytes. Dendricity may be measured with an Incucyte ZOOM® live cell imaging system available from Essen Bioscience, Ann Arbor, Mich. “Reduced dendricity” means that the total length of the dendrites is reduced. A suitable method measuring dendricity is disclosed in U.S. Provisional App. No. 62/050,008 filed by Hakozaki et al., on Sep. 12, 2014 and titled “Compositions and Methods for Inhibiting HMGB1 Activation of Melanocytes.”

“Dendrite” means a branched, tendril-like projection of a melanocyte that acts to transfer melanosomes from the melanocyte cell body to adjacent keratinocytes.

“Effective amount” means the amount of encapsulated nicotinamide riboside sufficient for the nicotinamide riboside to provide the desired skin benefit over the course of a treatment period. For example, in some instances, an effective of NR is an amount sufficient to provide a skin lightening benefit (e.g., improve the appearance of a hyperpigmented spot) over the course of a treatment period.

“Encapsulated” means that at least 80% of the surface area of a nicotinamide riboside particle is covered by an encapsulating agent. For example, at least 85%, 90%, 91%, 92%, 93%, 94%, 95%, 96%, 97, 98%, 99%, and ideally 100% of the surface area of an encapsulated NR particle is covered by the encapsulating agent.

“Water impermeable” refers to a material through which water and other fluids cannot pass absent catastrophic failure of the material (e.g., rupturing, tearing, breaking, melting, or dissolving).

“Generally recognized as safe” or “GRAS” refers to a material that complies with Sections 201(s) and 409 of the Federal Food, Drug, and Cosmetic Act, and the U.S. Food and Drug Administration's implementing regulations in 21 CFR 170.3 and 21 CFR 170.30, which require the premarket review and approval by the FDA of any use of a food substance, unless the substance is generally recognized, among qualified experts, as having been adequately shown to be safe under the conditions of its intended use either through scientific procedures or, for a substance used in food before 1958, through experience based on common use in food.

“Hyperpigmented” and “hyperpigmented skin” mean a localized portion of skin with relatively high melanin content. Examples of hyperpigmented skin include, but are not limited to age spots, melasma, chloasma, freckles, post inflammatory hyperpigmentation, sun-induced pigmented blemishes and the like.

“Improve the appearance of” means providing a measurable, desirable change or benefit in skin tone appearance or the appearance of a hyperpigmented spot, which may be quantified by a reduction in the Spot Area Fraction and/or an increase in L* value. Exemplary methods for determining these values are described in more detail in the Methods section below.

“L*a*b*” refers to the commonly recognized color space specified by the International Commission on Illumination (“CIE”). The three coordinates represent (i) the lightness of the color (i.e., L*=0 yields black and L*=100 indicates diffuse white), (ii) the position of the color between magenta and green (i.e., negative a* values indicate green while positive a* values indicate magenta) and (iii) the position of the color between yellow and blue (i.e., negative b* values indicate blue and positive b* values indicate yellow).

“Skin care agent” means a cosmetic agent for regulating and/or improving a skin condition. Some nonlimiting examples of regulating and/or improving a skin condition include improving skin appearance and/or feel by providing a smoother, more even appearance and/or feel; increasing the thickness of one or more layers of the skin; improving the elasticity or resiliency of the skin; improving the firmness of the skin; reducing the oily, shiny, and/or dull appearance of skin; improving the hydration status or moisturization of the skin; improving the appearance of fine lines and/or wrinkles; improving skin exfoliation or desquamation; plumping the skin; improving skin barrier properties; improving skin tone; reducing the appearance of spots, redness or skin blotches; and/or improving the brightness, radiancy, or translucency of skin. Skin care agents may incorporated in topical compositions for directed application to a target skin area, or incorporated into an ingestible composition such as a beverage and delivered to a target skin portion via the digestive and circulatory systems of the body.

“Skin care composition” means a cosmetic composition that includes at least one skin care agent (e.g., encapsulated NR particles) disposed in a dermatologically acceptable carrier.

“Skin tone” means the overall appearance of melanin in the skin caused by the systemic, rather than transient, synthesis of melanin. Skin tone is typically characterized over a relatively large area of the body. (e.g., the face, arm, chest, shoulder, abdomen or a substantial portion of one or more of these). An exemplary area for evaluating skin tone is about 100 mm² or more. Skin tone may be determined using a suitable image analysis technique. For example, overall lightness can be determined by using the L* coordinate in the L*a*b* color space (International Commission on Illumination). Chromophore mapping such as melanin mapping and melanin concentration may also be used as an indicator of overall skin tone.

“Skin tone agent” means a cosmetic agent intended to be applied to the skin for the purpose of effectuating a change in skin pigmentation.

“Skin lightening” means one or more of the following: overall lightening of basal skin tone, reduction in spot area or lightening of hyperpigmented regions, including age spots, melasma, chloasma, freckles, post inflammatory hyperpigmentation or sun-induced pigmented blemishes. Changes in skin lightening may be determined by visual grading and/or by measuring a change in L* value in a region of interest, for example, using a spectrophotometer or the like.

“Stable” means that a composition or ingredient retains a desired level of potency for the duration of a predetermined expiration period, as defined by generally accepted pharmaceutical or cosmetological protocols (e.g., good manufacturing practices (“GMP”)), or as promulgated by various trade conventions such as, for example, the United States Pharmacoepia Convention. For example, a stable encapsulated NR particle herein may exhibit less than 20% hydrolysis when placed in an aqueous solution at 15-40° C.±2° C. (e.g., 16° C., 18° C., 20° C., 22° C., 24° C., 26° C., 28° C., 30° C., 32° C., 34° C., 36° C., 38° C., 40° C., ±2° C.) for at least 1 hour (e.g., at least 2 hours, 5 hours, 8 hours, 12 hours, or even at least 24 hours). In some instances, the encapsulated particles herein may be stable in an aqueous solution at 15-40° C. for more than 1 day, 2 days, 3 days, 4 days, 5 days, 6 days, 7 days or even for more than 2weeks, 1 month, 2 months, 3 months, 4 months, 5 months, or even more than 6 months). Stability may be determined according to the Hydrolysis Test described in more detail below.

“Treatment period,” as used herein means the length of time and/or frequency that the encapsulated cosmetic agent is used. The treatment period may be a predetermined length of time and/or frequency, but need not necessarily be so.

“Water insoluble” refers to a material that does not readily dissolve in water (e.g., has a water solubility at 25-50° C. of less than 200 millimoles/liter, less than 100 millimoles/liter, less than 50 millimoles/liter or even less than 10 millimoles/liter).

### Skin Care Composition

The present skin care compositions include an effective amount of stable NR particles, a dermatologically acceptable carrier and, optionally, additional ingredients known for use in skin care compositions. The skin care compositions herein may be provided in various product forms that include, but are not limited to, solutions, suspensions, lotions, creams, gels, toners, sticks, sprays, aerosols, ointments, cleansing liquid washes and solid bars, pastes, foams, mousses, shaving creams, wipes, strips, patches, electrically-powered patches, hydrogels, film-forming products, facial and skin masks (with and without insoluble sheet). A skin care composition form may follow from the particular dermatologically acceptable carrier chosen, if present in the composition. Skin care compositions herein may be made using conventional methods for making cosmetic compositions.

In some instances, the skin care compositions herein include an amount of NR sufficient to reduce the level of HMGB1 protein released from keratinocytes, for example, by at least 10%, 15%, 20%, 25%, 30%, 35%, 40%, 45%, 50%, 55%, 60%, 65%, 70%, 75%, 80%, 85%, 90%, 95% or even 100%. It may be particularly desirable for the effective amount of NR to reduce HMGB1 protein levels and/or activity in stressed keratinocytes (i.e., keratinocytes exposed to a stressor such as ultraviolet radiation) to pre-stress levels or below, which could result in reduction of greater than 100%. HMGB1 protein level can be determined by using a conventional HMGB1 ELISA kit (e.g., the High Mobility Group Box 1 Protein (HMGB1) ELISA Kit available from IBL International as REF #ST51011) according to the manufacturer's instructions.

Reducing HMGB1 protein levels, and thus dendrite stimulation caused by HMGB1 protein may improve the appearance of hyperpigmented spots and/or overall skin tone. In some instances, the improvement may correspond to a positive change in L* value (i.e., a ΔL* value that is greater than 0, but typically less than 100) when the nicotinamide riboside is applied during a treatment period and/or at the end of a treatment period. In some instances, the ΔL* value may be from 0.1 to 10, from 0.2 to 5, from 0.3 to 3. Additionally or alternatively, the improvement in appearance may correspond to a reduction in Spot Area Fraction of at least 2% (e.g., from 2% to 100%, from 5% to 70%, from 10% to 40%, from 15% to 25%).

It is to be appreciated that reducing HMGB1 level is not considered “inhibiting melanin uptake” as that term is used when referring to the mechanism of action believed to be associated with niacinamide. In some instances, it may be desirable to ensure that the effective amount of NR does not inhibit (or promote) melanin uptake. In this way, it may be possible to provide improved skin appearance benefits by formulating compositions that include skin tone agents that function via different biological pathways.

### Stable NR Particles

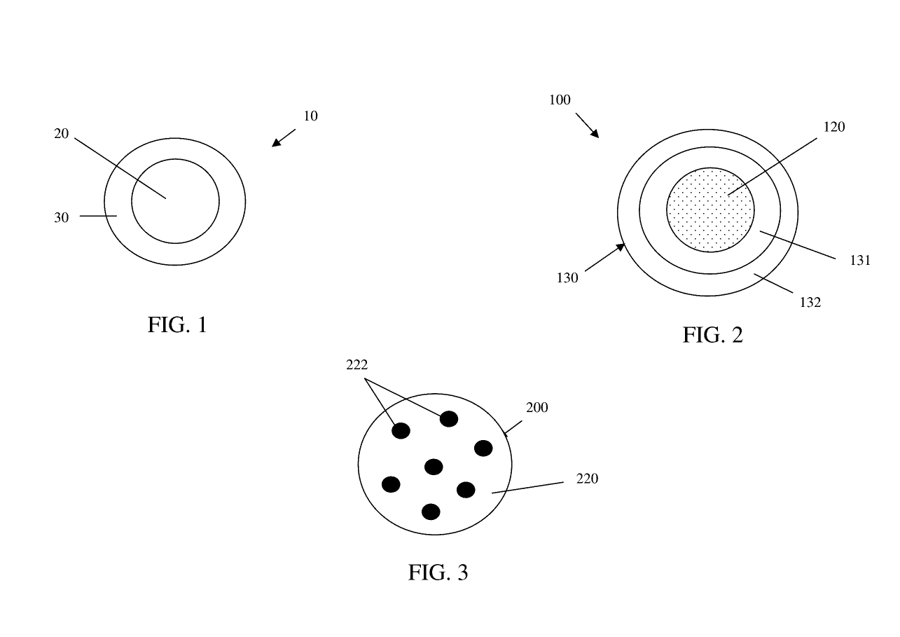

The stable NR particles herein include NR, and optional additional ingredients, coated with an encapsulating agent to provide encapsulated particles in a core-shell configuration, for example, as illustrated in FIGS. 1, 2 and 3, which are described in more detail below. The encapsulated particles may be present in the composition at an amount sufficient to provide from 0.0001% to 20% NR (e.g., from 0.001% to 15%, from 0.01% to 10%, or from 0.1% to 5%) by weight, based on the weight of the composition. The shell may comprise from 5% to 80% (e.g., from 10% to 40%) of the weight of the particle and have a total shell thickness of from 10 nanometers (“nm”) to 1 mm (e.g., between 10 nm and 500 micrometers (“μm”), 20 nm and 300 μm, 50 nm and 200 μm, 100 nm and 100 μm, 200 nm and 1 μm, 300 nm and 500 nm, or even between 300 nm and 400 nm). The core may include from 1% to 99% NR based on the weight of the particle. The encapsulated particles herein have a weight average particle size of less than 500 microns (μm) (e.g., less than 400 μm, 300 μm, 250 μm, 200 μm, 150 μm, 100 μm or even less than 50 μm) but typically larger than 1 μm (e.g., larger than 10 μm, 20 μm, 50 μm or even larger than 100 μm). Particles larger than 500 μm, or even larger than 300 μm, may be unsuitable for use in beverages because they tend to impart an undesirable gritty texture to the beverage. In some instances, particles larger than 100 μm may not be suitable for use in topical compositions because they can be harder to suspend in a cosmetic composition leading to a gritty and/or non-homogenous feel. On the other hand, particle sizes less than 1 μm have a surface area-to-volume ratio that undesirably favors increased hydration of the core relative to larger particles. And smaller particles may introduce undesirable processing difficulties and/or safety concerns.

In some instances, it may be desirable to communicate to a user that a benefit agent (e.g., NR) is present in a composition such as a beverage) by making the encapsulated particles visible. Visible particles may be provided by any suitable method known for imparting visibility to particles in a beverage, for example, by including GRAS pigments and/or dyes.

FIG. 1 is an illustration of an encapsulated particle 10 that includes a solid spherical core 20 surrounded by an encapsulating shell 30. The shell 30 includes an encapsulating agent and provides a water barrier between the core 20 and the external environment. As used herein, “water barrier” refers to a shell layer or material that prevents or at least inhibits water from hydrolyzing NR in the core and/or in an underlying layer. The water barrier may be water insoluble and/or water impermeable. In the example shown in FIG. 1, the shell 30 provides a water barrier around the NR-containing core 20, but can allow the NR to be released when the particle 10 or a composition containing the particle 10 is used as intended. The core 20 illustrated in FIG. 1 is spherical, but it is to be appreciated that the core 20 can be any shape, as desired. It may be desirable for the core 20 to be completely surrounded by the shell 30, as shown in FIG. 1, in order to adequately insulate the water-sensitive NR in the core 20 from contact with the water present in an aqueous composition. But it is to be appreciated that some of the particles in the compositions herein may have less than 100% of the core covered by the encapsulating agent.

In some instances, the encapsulated particles may include a shell comprising more than one layer of the same or different materials. For example, an encapsulated particle herein may comprise a multi-layer shell in which a first, outer layer functions as a water barrier by preventing or inhibiting water from penetrating the first layer and a second, inner layer that functions to scavenge any water that penetrates the first layer, thereby reducing the amount of water available to hydrolyze the NR in the particle. In some instances, the encapsulated particle may include multiple water barrier layers and/or NR-containing layers, for example, to provide an encapsulated particle that releases a desired amount of NR over a predetermined period of time (“controlled release particle”). The controlled release particle in this example may include an NR-containing core surrounding by alternating layers of water barrier and/or scavenging material and NR-containing material.

FIG. 2 is an illustration of an encapsulated particle 100 with a multi-layer shell 130 surrounding an NR-containing core 120. The NR in the core 120 may be solid (i.e., contains less than 5% liquid), dissolved in a miscible fluid or dispersed in an immiscible fluid. The multi-layer shell 130 includes a first, outer layer 132 and a second, inner layer 131. While not shown in FIG. 2, it is to be appreciated that the encapsulated particle 100 may, optionally, include one or more additional layers disposed around the first layer 132 and/or second layer 131. The first layer 132, second layer 131, and optional additional layers may be made from the same or different material and may provide the same or different functions, as desired. Each layer 131, and 132 of the multi-layer shell 130 may have the same or different thickness (e.g., between 1 nm and 500 μm, 10 nm and 300 μm, 50 nm and 100 μm, 100 nm and 50 μm, or even between 200 nm and 1 μm), as long as the NR in the encapsulated particle 100 is able to provide the desired skin care benefit.

In some instances, the encapsulated particles herein may include multiple cores surrounded by a continuous, unitary shell, for example, as illustrated in FIG. 3. FIG. 3 shows an encapsulated particle 200 that includes multiple NR-containing cores 222 surrounded by a unitary shell 220. Multi-core encapsulated particles like the one illustrated in FIG. 3 may be made using known processing techniques such as prilling, spray chilling, spray drying microfluidics, extrusion and loading a porous carrier.

Core

The encapsulated skin care agent herein includes a nicotinamide riboside containing core coated with an encapsulating agent. Nicotinamide riboside (CAS No. 1341-23-7) has the formula:

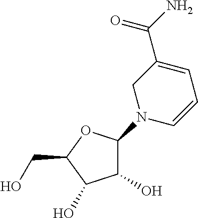 [SMILES: NC(=O)C1=CC=CN([C@@H]2O[C@H](CO)[C@@H](O)[C@H]2O)C1]

Some examples of nicotinamide riboside and its methods of manufacture are described in U.S. Pat. No. 8,106,184. As used herein, the term “nicotinamide riboside” includes derivatives of nicotinamide riboside (e.g., nicotinamide riboside chloride). Nicotinamide riboside may be obtained from ChromaDex, Inc., Irvine, Calif. The encapsulated particles contain at least 1% NR, based on the weight of the particle, but typically less than 90% (e.g., from 5% to 90%, 20% to 70% or even from 40% to 60%).

In addition to NR, the core may puonally include one or more other ingredients commonly included in cosmetic compositions (e.g., colorants, skin tone agents, skin anti-aging agents, nutritional supplements such as vitamins and minerals, anti-inflammatory agents, sunscreen agents, combinations of these and the like), provided that the additional ingredients do not undesirably alter the skin care benefit provided by the NR. The additional ingredients should be suitable for use in contact with human skin tissue without undue toxicity, incompatibility, instability, allergic response, and the like. Some nonlimiting examples of additional ingredients which may be suitable for use herein are described in U.S. Publication Nos. 2002/0022040; 2003/0049212; 2004/0175347; 2006/0275237; 2007/0196344; 2008/0181956; 2010/00092408; 2008/0206373; 2010/0239510; 2010/0189669; 2011/0262025; 2011/0097286; US 2012/0015004 US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

### Shell

The encapsulating agents herein form a film around the NR-containing core to provide a suitable water barrier between the NR-containing core and the external environment. The shell formed by the encapsulating agent may be frangible or pliable (e.g., plastic, elastic or plastoelastic), as long as the NR in the core and/or shell, e.g., when a multi-layer shell is used, is released as intended. It may be desirable to provide an encapsulating shell that releases the NR when the particle is subjected to the shearing and/or crushing force typically experienced during topical application of a cosmetic composition. Additionally or alternatively, it may be desirable to provide an encapsulating shell that releases the NR when the particle is exposed to one or more conditions typically found in the gastrointestinal tract of a human. Encapsulating agents that may be used herein are not particularly limited and can include any suitable GRAS material that provides a desirable combination of water barrier and NR release properties. Some non-limiting examples of encapsulating agents that may be suitable for use herein are chitin and chitosan; cellulose and cellulose derivatives such as cellulose acetate phthalate, hydroxypropyl methyl cellulose, carboxymethyl cellulose, enteric/aquateric coatings and mixtures thereof; silicates, phosphates, and borates; polyvinyl alcohol; polyvinyl acetate/polyvinyl alcohol blends; polyethylene glycols; linear and branched carbohydrates such as simple sugars (monosaccharides) and mixtures thereof, oligosaccharides (2-10 monosaccharide units), and polysaccharides (35 or greater monosaccharide units) and mixtures of these; carbohydrates that have been modified to improve their water resistance properties (e.g., by adding alkyl or aryl functionalities); waxes; oil-in-water emulsions comprising silicone oils, silicone gels, or silicone elastomers suspended in water; aqueous latex dispersions comprising film forming polymer particles of polyacrylate, polyurethanes, silicas, and silicones, which upon dehydration coalesce to make uniform, low permeability films. The encapsulating agents may optionally include plasticizers such as, for example, sorbitol, polyethylene glycol and polypropylene glycol to help achieve a more homogeneous, impermeable coating. Plasticizers when included in the encapsulating agent may be present at from 0.01% to 10% by weight, based on the weight of particle.

In some instances, the shell may be in the form a discrete, continuous layer of material that surrounds the core, for example, as illustrated in FIGS. 1 and 2. In some instances, the shell may be in form of a solid matrix in which particles of NR (solid or contained in a liquid) are homogeneously dispersed, for example, as illustrated in FIG. 3.

Dermatologically Acceptable Carrier

The compositions herein may include a dermatologically acceptable carrier (“carrier”) that provides a suitable matrix to store and deliver the encapsulated skin care agent and other optional ingredients. The phrase “dermatologically acceptable carrier”, as used herein, means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. Non-limiting examples include simple solutions (e.g., aqueous, organic solvent, or oil based), emulsions, and solid forms (e.g., gels, sticks, flowable solids, or amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil and oil-in-water-in-oil). The oil phase herein may comprise silicone oils, non-silicone oils such as hydrocarbon oils, esters, ethers, and the like, and mixtures thereof.

The aqueous phase typically comprises water. However, in some instances, the aqueous phase may comprise components other than water, including but not limited to water-soluble moisturizing agents, conditioning agents, anti-microbials, humectants and/or other water-soluble skin care actives. In one embodiment, the non-water component of the composition comprises a humectant such as glycerin and/or other polyols.

A suitable carrier is selected to yield a desired product form. Furthermore, the solubility or dispersibility of the components (e.g., extracts, sunscreen active, additional components) may dictate the form and character of the carrier. In one embodiment, an oil-in-water or water-in-oil emulsion is preferred.

Emulsions may further comprise an emulsifier. The composition may comprise any suitable percentage of emulsifier to sufficiently emulsify the carrier. Suitable weight ranges include from about 0.1% to about 10% or about 0.2% to about 5% of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986). Suitable emulsions may have a wide range of viscosities, depending on the desired product form.

The carrier may further comprise a thickening agent as are well known in the art to provide compositions having a suitable viscosity and rheological character.

### Optional Ingredients

The skin care compositions herein may include one or more optional ingredients known for use in topical skin care compositions, provided the optional ingredients do not unacceptably alter the desired benefits of the composition. The optional ingredients, when present, may be included at an amount of about 50%, 40%, 30%, 20%, 10%, 5%, or 3%, by weight of the composition, for example, at least about 0.001%, 0.01%, 0.1%, 0.2%, 0.5%, or 1%, by weight of the composition. Suitable ranges include any combination of the lower and upper limits including suitable ranges from about 0.1% to about 50%; from about 0.2% to about 20%; or from about 1% to about 10%, by weight of the composition.

The optional ingredients, when incorporated into the composition, should be suitable for use in contact with human skin tissue without undue toxicity, incompatibility, instability, allergic response, and the like. Nonlimiting examples of optional components include skin anti-aging agents, skin tone agents, anti-inflammatory agents, anti-acne actives, desquamation actives, anti-cellulite agents, chelating agents, flavonoids, tanning active, non-vitamin antioxidants and radical scavengers, hair growth regulators, anti-wrinkle actives, anti-atrophy actives, minerals, phytosterols and/or plant hormones, N-acyl amino acid compounds, antimicrobial or antifungal actives, and other useful skin care actives, which are described in further detail in U.S. Publication Nos. US2006/0275237A1 and US2004/0175347A1.

Methods of Making

The stable skin care agent herein may be made using conventional method of encapsulating a water soluble active to provide a stable particle for use in an aqueous composition. In particular, the encapsulated skin care agent herein may be made by applying one or more coatings of an encapsulating agent to an NR-containing core material such that less than 20% of the NR in the core is hydrolyzed after encapsulation. Such encapsulated particles can be produced in a variety of ways such as, for example, coacervation, polycondensation, interfacial polymerization, emulsion polymerization, solvent evaporation, solvent exchange, lyophilization, nanoprecipitation, spray drying, spray chilling, prilling, extrusion, and fluid bed coating. Some non-limiting examples of particle formation, encapsulation and/or coating techniques are disclosed in U.S. Pat. Nos. 5,550,119; 7,338,928; 6,790,821; 8,236,715; and 8,945,419; 9,029,083; 9,039,273 and U.S. Publication Nos. 2003/0207776; 2003/0232091; 2004/0096515; 2005/0276831; 2006/0078893; 2007/0054119; 2007/0092914; 2009/0197772; 2010/0104712; 2010/0158984; 20100159079; 2010/0213628; 2011/10268802; 2011/0269657; 2011/0143985; 2011/0143984; 2012/0015004; 2012/0077880; 2012/0077881; 2014/0065234; 2014/0220087; 2014/0178964; 2015/0099680; and 2015/0010600.

In some instances, NR powder particles can be directly coated with an encapsulation agent using a fluidized bed coating/drying operation, which results in particles with a solid core. For example, a Wurster brand fluidized bed coater or equivalent may be used to provide a continuous, unbroken coating around NR powder particles. In this example, the NR powder is sprayed with a suitable coating material (e.g., an aqueous solution of film forming polymers or a meltable, hydrophobic material that solidifies or crystallizes on the surface of the NR core). The spray-on encapsulation agent may be in the form of a suspension, emulsion or dispersion. The fluidized bed is operated such that the flux number of the fluid bed is between 3.5 and 7 (e.g., between 3.5 and 5.0) and the Stokes number is greater than 1 (e.g., between 10 and 1000 or between 100 and 1000). The flux number provides and estimation of the operating parameters of a fluidized bed to control coating within the bed, and the Stokes number is a measure of particle coalescence for describing the degree of mixing occurring to particles in the fluid bed. U.S. Pat. No. 6,790,821 to Wasserman, et al., describes how to determine flux number and Stokes number. The sprayed particles in the fluidized bed are then dried with dehumidified air maintained below the degradation temperature of the NR. The resulting coated particles should have a weight average particle size of between 20 and 800 microns.

Optionally, the NR may be mixed with inert materials and binders prior to the fluidized bed process to achieve a particle size that is appropriate for fluidization. For example, particles<20 micrometers are typically not appropriate for fluidization (they will elutriate out of the bed), and particles greater than 800 microns are not appropriate for fluidization (they will require high fluidization velocities).

The fluid bed mixer includes at least one coating zone where the encapsulation agent is applied. The coating zone involves the spraying of the encapsulation agent onto the fluidized particles. The bed may be fluidized with heated air. Spraying may be achieved via nozzles capable of delivering a fine or atomized spray of the encapsulation agent to achieve complete coverage of the particles. Typically, the droplet size from the atomizer is less than 2 times the particle size. This atomization can be achieved either through a conventional two-fluid nozzle with atomizing air, or alternatively by means of a conventional pressure nozzle. It may be desirable to position the nozzle above the fluidized height of the particles in the fluid bed to allow a vertical down spray of the coating mixture (i.e., a top spray configuration). The coating zone of the fluid bed may be followed by a drying zone and a cooling zone. It is to be appreciated that alternative arrangements are also possible to achieve the desired coated particles.

Typical conditions within a fluid bed apparatus include: (i) from 1 to 20 minutes of mean residence time; (ii) from 100 to 600 mm of depth of unfluidized bed; (iii) a droplet size of less than 2 times the size of the particles, (e.g., not more than 100 μm or 50 microns); (iv) from 150 to 1600 mm of spray height from the fluid bed plate or preferably 0-600 mm from the top of the fluid bed, (v) from 0.1 to 4.0 m/s of fluidizing velocity, preferably 1.0 to 3.0 m/s; and (vi) from 12 to 200° C. of bed temperature (e.g., 15 to 100° C.). Again, one of ordinary skill in the art will recognize that the conditions in the fluid bed may vary depending on a number of factors.

In some instances, NR powder may be dissolved in a miscible solvent, and droplets of the resulting NR-containing solution can be encapsulated using known chemical or physical encapsulation techniques, resulting in the formation of encapsulated particles with a liquid core. Some non-limiting examples of solvents that can dissolve NR are 3-methyl isoxazole, acetanilide, succinic anhydride, pyridazine, 1-methyl imidazole, salicylaldehyde, tetrahydrofurfuryl alcohol, 2-pyrolidone, 2-pyrrolidinone, isoxazole, dimethyl sulfone, tetramethylene sulfone, thiazole, thiourea, b-propiolactone, ethylene cyanohydrin, dimethyl sulfoxide, dimethyl sulfoxide, 1,3-triazole, diethylenetriamine, diethylenetriamine, dimethyl formamide, n,n-dimethylformamide, 2-chloropropenoic acid, acetonecyanhydrin, shellac, polyethylene oxide 4000, sorbitol and mixtures of these.

In some instances, NR powder can be dispersed in an immiscible solvent, and the dispersion can then be encapsulated using chemical or physical encapsulation techniques known in the art for encapsulation of lipophilic oils, resulting in the formation of encapsulated particles with a liquid core. Some non-limiting examples of immiscible solvents are mono, di- and tri-esters of C4-C24 fatty acids and glycerin; fatty acid esters of polyglycerol oligomers; polyalphaolefins, butyl oleate, hydrogenated castor oil, sucrose benzoate, dodecanoic acid, palmitic acid, stearic acid, Octadecanoic acid, monoester with 1,2,3-propanetriol; Dodecanoic acid, pentyl ester; Octanoic acid, nonyl ester; Pentadecanoic acid, ethyl ester; Hexadecanoic acid, methyl ester; Dodecanoic acid, 4-methylphenyl ester; Dodecanoic acid, 3-methylbutyl ester; Tetradecanoic acid, 1-methylethyl ester; Hexadecanoic acid; 1-Phenanthrenecarboxylic acid, hexarose; butyl oleate; hydrogenated castor oil; isopropyl myristate; castor oil; mineral oil; isoparaffin; capryllic triglyceride; soybean oil; vegetable oil; geranyl palmitate; silicones; polydimethylsiloxane; Heptadecane, isododecane; perfume raw materials with a Calculated log P (“C log P”) of greater than 5 using the C LOG P program available from Daylight Chemical Information Systems Inc., Irvine, Calif.

In some instances, an NR-solution (e.g., NR dissolved in a miscible, liquid) may be dispersed in a meltable immiscible solvent, which is then prilled or spray chilled to produce encapsulated particles. In a prilling operation, the melted suspension is dosed onto a centrifugal atomizer. The centrifugal atomizer generates atomized particles that are subsequently cooled in the air.

In some instances, solubilized NR can be pre-loaded into a porous carrier such as zeolites, precipitated silicas or lattice-network microspheres, and then encapsulated according to one of the aforementioned encapsulation techniques. In this way, the solubilized NR contained in the pores of the porous carrier is protected from hydrolysis by the lattice structure of the carrier and the encapsulation agent, which work cooperatively to hinder hydrolysis of the NR.

In some instances, the NR powder (either dissolved in a miscible solvent or dispersed in an immiscible carrier fluid) can be encapsulated in single or multiple shells using a microfluidic technique. To form a single shell, the NR-containing fluid and shell materials/precursors are pushed through a concentric nozzle, then emulsified into drop-in-a-drop (double emulsion) by a continuous phase fluid. By adding extra flow channels, additional shell layers can be formed to yield a microcapsule suspension that provides adequate hydrolysis stability to NR when dosed in a finished product formulation. U.S. Publication No. 2008/0213593 discloses microfluidic techniques that may be suitable for encapsulating the skin care agents herein.

In some instances, the encapsulated particles herein may be coated with a material to reduce the rate of leakage of NR from the particles when the particles are subjected to a bulk environment (e.g., storage and shipping). Some non-limiting examples of such materials include polyvinyl pyrrolidone homopolymer, and its various copolymers with styrene, vinyl acetate, imidazole, primary and secondary amine containing monomers, methyl acrylate, polyvinyl acetal, maleic anhydride; polyvinyl alcohol homopolymer, and its various copolymers with vinyl acetate, 2-acrylamide-2-methylpropane sulfonate, primary and secondary amine containing monomers, imidazoles, methyl acrylate; polyacrylamides; polyacrylic acids; microcrystalline waxes; paraffin waxes; modified polysaccharides such as waxy maize or dent corn starch, octenyl succinated starches, derivatized starches such as hydroxyethylated or hydroxypropylated starches, carrageenan, guar gum, pectin, xanthan gum; modified celluloses such as hydrolyzed cellulose acetate, hydroxy propyl cellulose, methyl cellulose, and the like; modified proteins such as gelatin; hydrogenated and non-hydrogenated polyalkenes; fatty acids; hardened shells such as urea crosslinked with formaldehyde, gelatinpolyphosphate, melamine-formaldehyde, polyvinyl alcohol crosslinked with sodium tetraborate or gluteraldehyde; latexes of styrene-butadiene, ethyl cellulose; and mixtures thereof.

Methods of Use

Various methods of treatment, application, regulation, or improvement may utilize the aforementioned compositions. In some instances, the methods herein include identifying a target portion of skin (e.g., a facial skin surface such as the forehead, perioral, chin, periorbital, nose, and/or cheek) in need of treatment (e.g., skin that includes visible pigmentation disorders, fine line or wrinkles or other undesirable skin conditions) and/or where treatment is desired, and applying a safe and effective amount of the skin agents herein, which may be incorporated into a suitable cosmetic composition, to the target portion of skin. In some instances, the target portion of skin may not exhibit visible signs of a skin condition, but a user (e.g., a relatively young user) may still wish to target such an area of skin if it is one that typically develops skin disorders later in life (e.g., skin surfaces that are typically not covered by clothing, such as facial skin surfaces, hand and arm skin surfaces, foot and leg skin surfaces, and neck and chest skin surfaces). In this way, the present methods and compositions may be used as a preventative measure. Skin care compositions containing an effective amount of the present skin care agent may be applied to the target skin portion and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed.

The treatment period is ideally of sufficient time for the NR to improve the appearance of the target portion of skin, which may correspond to a reduction in the size of hyperpigmented spot and/or an increase in lightness. The treatment period may last for at least 1 week (e.g., about 2 weeks, 4 weeks, 8 weeks, or even 12 weeks). In some instances, the treatment period will extend over multiple months (i.e., 3-12 months) or multiple years. In some instances, a cosmetic composition containing an effective amount of nicotinamide riboside may be applied most days of the week (e.g., at least 4, 5 or 6 days a week), at least once a day or even twice a day during a treatment period of at least 2 weeks, 4 weeks, 8 weeks, or 12 weeks.

The cosmetic compositions herein may be applied locally or generally. In reference to application of the composition, the terms “localized”, “local”, or “locally” mean that the composition is delivered to the targeted area (e.g., a hyperpigmented spot or portion thereof) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied more generally or broadly to one or more skin surfaces. In certain embodiments, the compositions herein may be used as part of a multi-step beauty regimen, wherein the present composition may be applied before and/or after one or more other compositions.

In some instances, the method herein includes identifying a target portion of facial skin in need of treatment and orally ingesting a safe and effective amount of nicotinamide riboside over the course of a multi-day, or multi-week, or multi-month, or multi-year treatment period. Nicotinamide riboside may be incorporated into a suitable oral composition. The target portion of facial skin is any portion of facial skin (e.g., periorbital, cheek, chin, perioral, nose, forehead, etc.) where improvement in the appearance of skin is desired (e.g., reduction in size and/or increase in lightness of hyperpigmented spot). In some instances, the target portion of skin may not exhibit a visible sign of a skin condition, but a user (e.g., a relatively young user) may still wish to target such an area of skin if it is one that typically develops skin conditions later in life (e.g., skin surfaces that are typically not covered by clothing, such as facial skin surfaces, hand and arm skin surfaces, foot and leg skin surfaces, and neck and chest skin surfaces). In this way, the present compositions may be used as a preventative measure for skin pigmentation disorders.

The oral composition may be ingested one time per day, two times per day, three times per day (e.g., around each meal), four times per day or more during the treatment period. The daily dosage of nicotinamide riboside may be greater than 100 mg, preferably greater than 250 mg, more preferably greater than 300 mg, most preferably greater than 400 mg or 500 mg. The weekly dosage may be greater than 2100 mg/week, 2800 mg/week, or 3500 mg/week. The daily dosage may be provided in a single unit dosage form (e.g., a single pill, capsule or tablet) or may be provided in smaller unit dosage forms if the oral composition is intended to be taken more than once per day.

The treatment period is ideally of sufficient time for the nicotinamide riboside to provide an improvement in the appearance of skin (e.g., an improvement of one or more pigmented spots in the target portion of facial skin), which may correspond to a reduction in the size of a pigmented spot. The treatment period may be at least 3 weeks, preferably at least 4 weeks, more preferably at least 6 weeks, most preferably at least 8 weeks. In some instances, the treatment period will extend over multiple months (e.g., 1, 2, 3, 4, 5, 6, 7, 8, 10, 12 or more months) or multiple years (e.g., 1, 2, 3, 4, 5 or more years). Notably, the inventors discovered that within 2 weeks or less after cessation of the treatment period, there was a regression to baseline in the appearance of the pigmented spots within 2 weeks, or possibly less. Therefore, in some instances, it is desirable that the daily dosage is uninterrupted during the treatment period, or interrupted for less than 2 weeks, or less than 1 week, or less than 5 days, or less than 3 days.

### Methods

Imaging Method

This method provides a means for capturing a reproducible and analyzable image for determining L*a*b* values and Spot Area Fraction. It is to be appreciated that any suitable image capture device along with imaging software and other associated ancillary equipment (e.g., computer and lights) which are equivalent to those described in this method may be used. The imaging system in this method incorporates a FUJI-S2 Pro brand CCD SLR digital camera which delivers a 6 megapixel uncompressed image (BMP) and a raw image file (RAF). Prior to taking a photograph, the test subject is illuminated with a JTL 1000 W flash through two linear polarizers in crossed axis orientation. A chart containing Munsell Color Standard Neutral N2-N9.5 are captured in every image for standardization and color correction purposes.

In preparation for image capture, test subjects are required to wash their face and wait for at least 15 minutes to let their face dry. The hair of the subject is covered with a hairnet and the head and shoulders of the subject are covered with a black cloth. All jewelry that can be seen in an image area of interest is removed. The subjects are equilibrated in a control room at 20-25° C. and 40-60% relative humidity for 30 minutes. Next, each subject is suitably positioned, in front of the camera and one or more images of each side of the face are captured. The captured image(s) are then processed by converting the raw image to a .jpg file format.

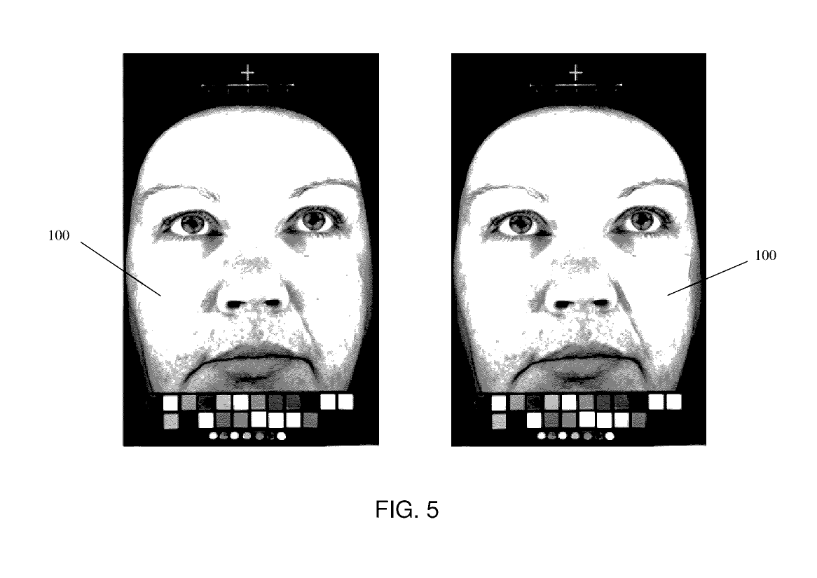

Next, the .jpg format image is analyzed by a computer with suitable image analysis software. In some instances, it may be desirable to analyze only a portion of the image (i.e., a region of interest (“ROI”)). The ROI may be “masked,” for example, as shown in FIG. 5, using image editing software such as Photoshop® or Image J® brand software. The masked region (e.g., cheek 100 in FIG. 5) can then be isolated and analyzed as a separate image. It is to be appreciated that the image need not necessarily be masked for suitable analysis, and in some instances the entire image may be analyzed. In some instances, it may be desirable to reduce the size of the image, mask and/or region of interest by several pixels (e.g., between 5 and 15 pixels) around the outer edge of the image where some shadowing may occur.

Since color may be perceived as being relative, depending on, for example, which instruments and/or imaging system is used, it can be important to color correct the image or region of interest for each subject using a suitable color correction technique (e.g., according to International Color Consortium standards and practices), which helps make the color determination by the system less instrument specific. The RGB values in the captured images, which are device dependent, are converted to L*a*b* values. The L*a*b* values can be calculated using a suitable RGB conversion tool (e.g., software installed on the computer or a suitable conversion tool found online). The conversion from RGB values to L*a*b* values can be performed on the entire image, a ROI or on one or more individual pixels. The resulting L*a*b* values may be averaged to provide average values for the image or a region of interest.

Spot Area is the total area of spots (in pixels) detected in the desired ROI. Spots are detected by comparison of localized detection of lower gray density objects from higher gray density background in the desired channel of the RGB color space. The detected objects are further classified by shape and size.

Spot Area Fraction (“SAF”) is calculated as the ratio of the area occupied by hyperpigmented spots to the target skin area multiplied by 100 (i.e., Spot Area/masked area*100). The changes in SAF indicate the change in area occupied by hyperpigmented spots over time (“ΔSAF”) (e.g., SAF_{final}−SAF_{baseline}) or relative to a control (“ΔΔSAF”) (e.g., ΔSAF_{test composition}−ΔSAF_{vehicle control}). A lower value reflects smaller and/or fewer hyperpigmented spots.

Hydrolysis Test

The Hydrolysis Test provides a method for determining the stability of NR. In particular, this method can be used to determine the amount of NR that is hydrolyzed when incorporated into an aqueous composition.

Sample Preparation

If the NR particles are not in a suitable aqueous vehicle (i.e., a skin care product or a vehicle that simulates an aqueous skin care product), mix the NR particles into a suitable aqueous vehicle. Table 1 below is provided as an example of ingredients that can be combined to form a suitable aqueous vehicle. The NR particles may be added at any amount desired, but are typically included at from 0.1% to 5% w/v. The ingredients may be combined using conventional methods of making skin care compositions. After the skin care composition is made, weigh 0.1 g of the skin care composition into a polypropylene conical centrifuge tube, and dilute with 25 mL of a diluent. The diluent is made from 5% (v/v) 5 mM ammonium formate, 0.025% (v/v) formic acid in Milli-Q water and 95% (v/v) acetonitrile. Vortex or homogenize as needed to disperse the product formulation in the diluent. Using a syringe, filter a sufficient amount of the sample into an autosampler vial for HPLC analysis. Prepare standard stock solutions in Milli-Q water for calibration. Dilutions are made into diluent to cover a range of approximately 5-350 μg/mL analyte in solution for calibration curves.

TABLE 1

| Component | % |
|---|---|
| Phase A | |
| water | qs |
| glycerol | 3.00 |
| disodium EDTA | 0.10 |
| Phase B | |
| Isopropyl Isostearate | 1.33 |
| Isohexadecane | 3.00 |
| cetearyl glucoside | 0.20 |
| cetyl alcohol | 0.32 |
| tocopherol acetate | 0.50 |
| PEG-100 stearate | 0.10 |
| stearyl alcohol | 0.48 |
| behenyl alcohol | 0.40 |
| ethyl paraben | 0.20 |
| propyl paraben | 0.10 |
| polymethylsilsesquioxane | 0.25 |
| Phase C | |
| polyacrylamide/C13-14 | 2.00 |
| isoparaffin/laureth-7 | |
| Phase D | |
| benzyl alcohol | 0.25 |
| dimethicone/dimethiconol | 2.00 |

HPLC Conditions

An Alliance 2695 brand HPLC system with 996 PDA detector (Waters, Milford, Mass.) or equivalent with the separation mode set to hydrophilic interaction chromatography (HILIC) is used as the chromatographic system. Inject 5 microliters of the diluted formulation samples or calibration standards into the column. Nicotinamide riboside is separated from other components in the product on a SeQuant ZIC-Hilic (4.6×100 mm; 5 micron particle size) stationary phase. Hold the column temperature at 30° C. The mobile phase is: (A) 5 mM ammonium formate with 0.025% (v/v) formic acid in Milli-Q water; and (B) a mixture of 95% acetonitrile and 5% mobile phase (A) also with 0.025% (v/v) formic acid. Begin the gradient at 100% (B) and hold for 3 minutes. Next, use 60% (B) until 16 minutes, and hold for 3 minutes before returning to the starting condition of 100% (B). The entire chromatographic run should take about 24 minutes using a flow rate of 1.0 mL/min. Table 2 shows the times and gradients used in the test. The diode array detector is set to scan wavelengths of 205-350 nm. Chromatograms are extracted at 260 nm. Retention time is approximately 12.6 minutes. Quantitation is performed using Chromeleon v.7.2 or equivalent chromatography data system software package. A linear curve fit of the response of the calibration standards is used to determine analyte in solution levels. Results are expressed as weight percent (w/w %) once corrected for the dilution factor and weight of formulation aliquot.

TABLE 2

| Time | | |
|---|---|---|
| (mm) | % A | % B |
| 0 | 0 | 100 |
| 3 | 0 | 100 |
| 16 | 40 | 60 |
| 19 | 40 | 60 |
| 24 | 0 | 100 |

### EXAMPLES

### Example 1

### Exemplary Cosmetic Compositions

Table 1 provides examples of topical skin care compositions suitable for use with the methods herein. The compositions are made by blending the A phase components with a suitable mixer (e.g., Tekmar RW20DZM) and heating to a temperature of 70-80° C. and maintaining the temperature while stirring. Separately, blend the B phase components with a suitable mixer and heat to 70-75° C., maintaining temperature while mixing. Phase B is added to Phase A while mixing well to emulsify. The emulsion is then milled using a suitable mill (e.g., Tekmar T-25) for 5 minutes. When the emulsion is at 60° C., phase C is added while continuing to mix. At 40° C., the ingredients of phase D and E are added to the emulsion. The emulsion is then milled using a suitable mill (Tekmar T-25) for 5 minutes resulting in a uniform product.

TABLE 1

| | Component | | | | | |
|---|---|---|---|---|---|---|
| | 1 | 2 | 3 | 4 | 5 | 6 |
| | % | | | | | |
| Phase A | | | | | | |
| water | qs | qs | qs | qs | qs | qs |
| glycerol | 5.00 | 7.00 | 3.00 | 10.00 | 5.00 | 15.00 |
| disodium EDTA | 0.10 | 0.05 | 0.10 | 0.10 | 0.05 | 0.10 |
| Phase B | | | | | | |
| Isopropyl Isostearate | 5.00 | 2.50 | 1.33 | 2.50 | 5.00 | 2.50 |
| Isohexadecane | 1.00 | 1.50 | 3.00 | 1.00 | 3.00 | 5.00 |
| Distearyldimonium Chloride | 0.00 | 0.50 | 1.00 | 1.50 | 0.00 | 1.50 |
| Steareth-2 | 0.50 | 2.00 | 1.00 | 1.00 | 1.50 | 3.00 |
| cetyl alcohol | 0.25 | 0.50 | 0.32 | 0.50 | 1.00 | 0.40 |
| tocopherol acetate | 0.00 | 0.50 | 0.50 | 0.50 | 0.25 | 1.00 |
| Steareth-21 | 0.50 | 1.00 | 0.40 | 0.80 | 1.25 | 2.00 |
| stearyl alcohol | 0.70 | 1.50 | 2.00 | 2.25 | 3.00 | 4.50 |
| behenyl alcohol | 0.80 | 1.00 | 0.40 | 0.60 | 1.50 | 0.60 |
| ethyl paraben | 0.20 | 0.20 | 0.20 | 0.20 | 0.20 | 0.20 |
| propyl paraben | 0.10 | 0.10 | 0.10 | 0.10 | 0.10 | 0.10 |
| polymethylsilsesquioxane | 1.25 | 2.50 | 2.00 | 0.50 | 0.25 | 1.50 |
| Phase C | | | | | | |
| Polyethylene | 1.50 | 1.00 | 1.50 | 2.00 | 1.25 | 1.00 |
| Phase D | | | | | | |
| Water | 5.00 | 10.00 | 10.00 | 5.00 | 10.00 | 15.00 |
| Encapsulated Nicotinamide | 2.00 | 5.00 | 5.00 | 2.50 | 4.00 | 7.00 |
| Riboside (% w/v) | | | | | | |
| dexpanthenol | 0.25 | 0.50 | 0.50 | 2.00 | 1.00 | 2.00 |
| Phase E | | | | | | |
| benzyl alcohol | 0.25 | 0.25 | 0.25 | 0.25 | 0.25 | 0.25 |
| dimethicone/dimethiconol | 0.5 | 1.00 | 2.00 | 0.25 | 2.00 | 2.00 |

Beverage

This example illustrates the use of encapsulated NR particles for use in a beverage. The encapsulated NR particles are in a core-shell configuration and have a particles size of between 100-300 μm. The liquid core contains NR dissolved in a suitable solvent (e.g., sorbitol or propylene glycol) to provide a 5% solution. Alternatively, the NR may be dispersed in an immiscible GRAS liquid such as glycerin or olive oil to provide a liquid core. The liquid core is then encapsulated using a fluidized bed coater and dried to yield particles suitable for use in a beverage. The NR particles are then incorporated into a beverage using conventional techniques for making beverages.

### Example 2

### Melanosome Uptake Assay

This example utilizes a Melanosome Uptake Assay to compare the ability of nicotinamide riboside and niacinamide to inhibit melanosome uptake into keratinocytes as compared to a vehicle control (i.e., a composition identical to the test composition and positive control except it does not include niacinamide or nicotinamide riboside).

Carboxyfluorescein diacetate (“CFDA”) (available from Sigma, St. Louis, Mo.) labeled melanosomes were prepared by incubating CFDA dye in SKMEL-188 culture cells (available from Sloan Kettering Institute) for 2 days at 37° C. in a THERMO SCIENTIFIC FORMA brand CO₂ incubator (available from Fisher Scientific, Waltham, Mass.). On day 3 of the test, melansomes were isolated from SKMEL-188 cells by step density centrifugation with sucrose solutions layered with different densities, which is well known in the art. Melanosomes were taken from the 1.6 M-2.0 M sucrose layers. The isolated melanosomes were placed in each well of a 6-well plate along with human neonatal keratinocytes (available from Thermo) (approximately 50,000 keratinocytes/well). 2 ml of the appropriate medium (i.e., the test composition, the positive control or the vehicle control) was added to each of the wells to produce 3 test wells (i.e., 3 replicates of each of three composition tested). The test composition was made by adding nicotinamide riboside chloride powder (available from Chromadex, Irvine, Calif.) to EPILIFE brand keratinocyte medium to produce a solution of 0.0025 w/v % nicotinamide riboside. The positive control was made by adding niacinamide to EPILIFE brand keratinocyte medium to produce a solution of 0.0025 w/v % niacinamide. Unmodified keratinocyte medium was used as a vehicle control.

The resulting test plates were incubated for two days in EPILIFE brand keratinocyte medium. On day 6 of the test, the keratinocytes were detached from the plates using trypsin and fluorescent-label counted by flow cytometry using an LSRFortessa brand flow cytometer (available from Becton Dickinson, NJ). The percentage of cells that had fluorescence (from CFDA label) was used as a metric to measure incorporation of melanosome uptake. Keratinocytes containing detectable levels of CFDA were counted as a fraction of all keratinocytes passing through the flow cytometer (indicated as % uptake in Table 2). The higher the percentage, the higher level of melanosome uptake into the keratinocytes.

Table 2 illustrates the results of the test. As shown in Table 2, the positive control appears to inhibit the rate of melanosome uptake compared to the vehicle control, which was expected. Surprisingly, the nicotinamide riboside appears to increase the rate of melanosome uptake compared to the vehicle control, which was not expected and which might initially suggest that: 1) nicotinamide riboside could worsen the appearance of pigmented spots; and 2) nicotinamide riboside, while an analogue of niacinamide, does not have all the same mechanisms of action as niacinamide.

TABLE 2

| | | Rate |
|---|---|---|
| | | of Melanosome Uptake |
| Sample | % Uptake | Versus Control |
| Vehicle Control | 36 | 100% |
| Positive Control (0.0025 w/v % | 26 | 72% |
| niacinamide) | | |
| Nicotinamide Riboside 0.0025 | 67 | 186% |
| w/v % | | |

### Example 3

### In Vitro UV Stress Test

This example compares the ability of nicotinamide riboside and niacinamide to reduce the amount of HMGB1 released from keratinocytes subjected to stress from ultraviolet (“UV”) radiation.

Human neonatal keratinocytes (available from Thermo) were placed in each well of 4 12-well plates. Each well also contained 2 ml of EPILIFE brand keratinocyte medium. The plates were incubated at 37° C. in a CO₂ incubator until cell confluency reached 70%. At this point, the cells, except for the negative control, were exposed to 15 mJ/cm² UVB (i.e., UV radiation with a wavelength of from 315-280 nm) in a BIO-SUN brand UV irradiating system (available from Vilber Lourmat, France). After UVB exposure, the keratinocyte medium in each well was replaced with an appropriate medium (i.e., niacinamide medium, nicotinamide riboside medium or a control medium) to produce the test plates. The test plates were incubated for 24 hours at 37° C. in a CO₂ incubator, after which the medium in each cell was removed and the HMGB1 level measured using a conventional HMGB1 ELISA kit (REF #ST51011, available from IBL International, Canada) according to the manufacturer's instructions.

The test media were made by adding either niacinamide or nicotinamide riboside to EPILIFE brand keratinocyte medium to produce a 0.001 w/v % solution. The control medium was unmodified keratinocyte medium.

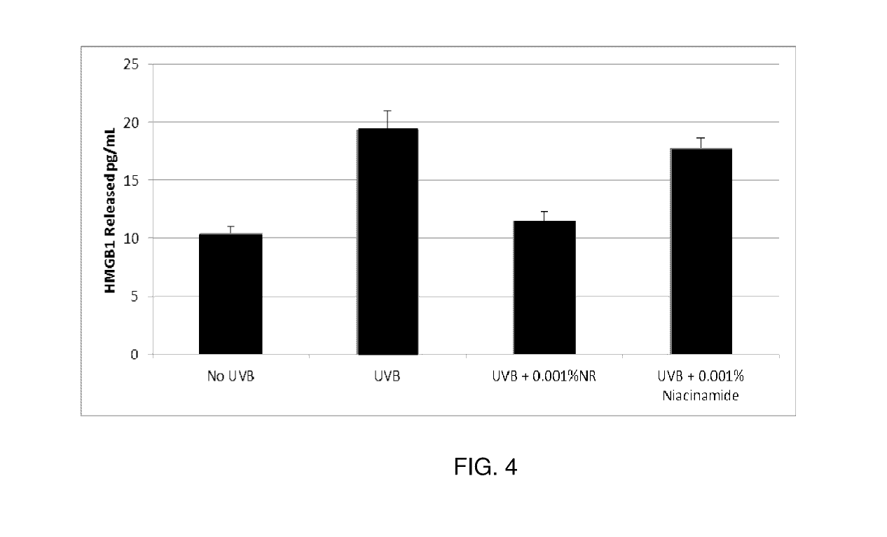

Table 3 illustrates the results of the test. As shown in Table 3, the untreated cells exposed to UVB radiation released more HMGB1 than the untreated cells that were not exposed to UVB, which is expected. Treating cells with niacinamide appears to have had no significant effect on the amount of HMGB1 released by keratinocytes exposed to UVB radiation when compared to the untreated UVB exposed cells. Surprisingly, the UVB exposed, nicotinamide riboside treated keratinocytes released less HMGB1 than the untreated, UVB-exposed keratinocytes. The results of Table 3 are illustrated in FIG. 4. The p-values shown in Table 3 are student's T-test, 2-sided, equal variance. P-values of less than 0.05 are considered statistically significant.

TABLE 3

| | HMGB1 Released | p-value (vs. UVB |
|---|---|---|
| Treatment | (pg/ml) | exposed, untreated cells) |
| No UV exposure, | 10.5 | <0.05 |
| untreated (negative | | |
| control) | | |
| UV exposure, untreated | 19.4 | 1 |
| (positive control) | | |
| UV exposure + 0.001% | 11.5 | <0.05 |
| Nicotinamide Riboside | | |
| Chloride | | |
| UV exposure + 0.001% | 17.8 | 0.24 |
| Niacinamide | | |

### Example 4

### Clinical Study

This example demonstrates the ability of a cosmetic composition comprising nicotinamide riboside to improve the appearance of a hyperpigmented spot and lighten skin. Composition #3 from Table 1 was used in this study.

The clinical study in this example is a 9-week, randomized, double-blinded, split-face, round robin study, which includes a 1 week normalization period and an 8 week test product usage period. The cosmetic compositions tested in the clinical study included a test composition comprising nicotinamide riboside (i.e., Composition #3 from Table 1) and the control composition set forth in Table 4, which is an oil-in-water emulsion similar to conventional moisturizing lotions/creams. The control composition was made using conventional methods known in the art for making such compositions.

TABLE 4
Control Composition

| Component | % |
|---|---|
| Phase A | |
| water | qs |
| glycerol | 3.00 |
| disodium EDTA | 0.10 |
| Phase B | |
| Isopropyl Isostearate | 1.33 |
| Isohexadecane | 3.00 |
| cetearyl glucoside | 0.20 |
| cetyl alcohol | 0.32 |
| tocopherol acetate | 0.50 |
| PEG-100 stearate | 0.10 |
| stearyl alcohol | 0.48 |
| behenyl alcohol | 0.40 |
| ethyl paraben | 0.20 |
| propyl paraben | 0.10 |
| polymethylsilsesquioxane | 0.25 |
| Phase C | |
| polyacrylamide/C13-14 | 2.00 |
| isoparaffin/laureth-7 | |
| Phase D | |
| benzyl alcohol | 0.25 |
| dimethicone/dimethiconol | 2.00 |

Asian females aged 25 to 55 years old and having relative dark skin tone (L*<60, by Chromameter CR400) and a suitable number of hyperpigmented spots were selected to participate in the study. Prior to application of a test or control composition, the test subjects washed their face with OLAY DEEP PURIFY CLEANSER brand facial cleanser. After washing, the test product was applied to one side of the test subject's face, and the vehicle control was applied to the other side of the subject's face. This was done twice per day (morning/evening) during the test period. Dosage was 0.5 g per split face (forehead to jawline˜4 mg/cm²). Measurements were taken at the start of the test period (baseline) and after 2, 4 and 8 weeks of treatment. Digital images were captured and analyzed for changes in L* value and SAF according the Imaging method described above. The data were statistically analysed with a known Mixed Model (e.g., available from SAS Institute, Cary, N.C., U.S.A.) for repeated measures with the subject effect fitted as random, and the other effects (treatment, side (left and right), week, treatment-by-week interaction, age, baseline) fitted as fixed. Values are considered statistically significant if the p-value is less than or equal to 0.05.

The results of the clinical study are illustrated in Tables 5 and 6. Table 5 shows the change in lightness values (ΔL* value) for the test composition relative to the control and baseline values at weeks 2, 4, and 8 for each composition. Baseline values for all test subjects were measured on Day 0 and averaged to provide a common baseline for use in the test. Table 6 shows the change in SAF (“ΔSAF”) observed at weeks 2, 4, and 8 for each composition. As shown in Tables 5, treatment with the test composition lightened the skin at week 8 (positive ΔL* value) relative to the baseline value, and lightened the skin better than the control at weeks 2, 4 and 8. As shown in Table 6, treatment with the test composition consistently reduced SAF at weeks 2, 4 and 8 relative to the baseline and reduced SAF more than the control composition, which did not appear to provide any significant reduction in SAF.

TABLE 5

| | | | | ΔL* from | | ΔL* from | |
|---|---|---|---|---|---|---|---|
| | N | Composition | L* Value | Baseline | p-value | Control | p-value |
| Baseline | 42 | — | 57.326 | — | — | — | — |
| Week 2 | 41 | Vehicle | 56.899 | −0.427 | <0.0001 | — | — |
| Week 4 | 41 | Control | 56.679 | −0.648 | <0.0001 | — | — |
| Week 8 | 41 | | 57.000 | −0.327 | <0.0001 | — | — |
| Week 2 | 41 | Test | 57.327 | 0.001 | 0.9986 | 0.428 | <0.0001 |
| Week 4 | 41 | Composition | 57.368 | 0.041 | 0.6788 | 0.689 | <0.0001 |
| Week 8 | 41 | (5% NR) | 57.817 | 0.491 | <0.0001 | 0.817 | <0.0001 |

TABLE 6

| | | | | ΔSAF | | ΔSAF | |
|---|---|---|---|---|---|---|---|
| | | | | from | | from | |
| | N | Composition | SAF (%) | Baseline | p-value | Control | p-value |
| Baseline | 42 | — | 6.755 | — | — | | |
| Week 2 | 41 | A (Control) | 6.830 | −0.075 | 0.4930 | — | — |
| Week 4 | 41 | A (Control) | 6.578 | −0.176 | 0.1669 | — | — |
| Week 8 | 41 | A (Control) | 6.665 | −0.090 | 0.4757 | — | — |
| Week 2 | 41 | C (5% NR) | 6.234 | −0.520 | <0.0001 | −0.596 | <0.0001 |
| Week 4 | 41 | C (5% NR) | 5.820 | −0.935 | <0.0001 | −0.758 | <0.0001 |
| Week 8 | 41 | C (5% NR) | 5.579 | −1.176 | <0.0001 | −1.086 | <0.0001 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition, comprising:
a. an effective amount of a stable skin care agent comprising a plurality of encapsulated particles in a core-shell configuration, wherein the core comprises nicotinamide riboside (NR) and the shell comprises an encapsulating agent; and
b. a dermatologically acceptable carrier.

2. The skin care composition of claim 1, wherein the NR is present at about 0.001% to about 20% by weight based on the weight of the skin care composition.

3. The skin care composition of claim 1, wherein less than 20% of the encapsulated NR is hydrolyzed according to the Hydrolysis Test.

4. The skin care composition of claim 1, wherein the shell is at least one of water impermeable and water insoluble.

5. The skin care composition of claim 1, further comprising a weight average particle size of between about 1 and about 500 microns.

6. The skin care composition of claim 5, wherein the core is a liquid core and the NR is dissolved in a miscible solvent.

7. The skin care composition of claim 6, wherein the core is a liquid core and the NR is dispersed in an immiscible fluid.

8. The skin care composition of claim 1, wherein the core comprises a porous carrier with the NR disposed thereon.

9. The skin care composition of claim 8, wherein the porous carrier is selected from zeolites, precipitated silicates, microspheres and combinations thereof.

10. The skin care composition of claim 1, wherein the encapsulated particle comprises two or more cores surrounded by a unitary shell.

11. The skin care composition of claim 1, wherein the encapsulated particle comprises one core surrounded by two or more shells.

12. The skin care composition of the claim 1, wherein the NR reduces a level of HMGB1 protein released by keratinocytes exposed to an environmental stressor by at least 10%.

13. The skin care composition of claim 12, wherein the environmental stressor is ultraviolet radiation.

14. A method of lightening skin, comprising:
a. identifying a target portion of skin where skin lightening is desired; and
b. topically applying a stable, encapsulated skin care agent to the target portion of skin during a treatment period, wherein the skin care agent comprises an effective amount of NR encapsulated by an encapsulating agent, and the treatment period is sufficient for the NR to lighten the target portion of skin.

15. The method of claim 14, wherein the skin lightening corresponds to a positive change in L* value.

16. The method of claim 15, wherein the positive change in L* value is at least 0.1.

17. A method of improving the appearance of a hyperpigmented spot, comprising:
a. identifying a target portion of skin that includes a hyperpigmented spot; and
b. topically applying a stable, encapsulated skin care agent to the target portion of skin during a treatment period, wherein the skin care agent comprises an effective amount of NR encapsulated by an encapsulating agent, and the treatment period is sufficient to allow the NR to reduce the hyperpigmented spot area relative to a baseline hyperpigmented spot area.

18. The method of claim 17, wherein the reduction in spot area corresponds to a reduction in Spot Area Fraction (SAF).

19. The method of claim 18, wherein the reduction in SAF corresponds to at least one of a ΔSAF and a ΔΔSAF of at least 0.2.

20. The method of claim 19, wherein the reduction in at least one of ΔSAF and ΔΔSAF is at least 5%.
